(19) United States
(12) Patent Application Publication
(10) Pub. No.: US 2026/0147864 A1
(43) Pub. Date: May 28, 2026

(54) Biometric-Authenticated Wearable Health Monitoring System for Remote Patient Care and Sentiment-Based Analysis

(71) Applicants: Padmanabaiah Srirama, Glen Allen, VA (US); Shankar Yalamanchili, Montgomerry, AL (US); Yashas Sai Chigurupati, O'Fallon, MO (US); Jesu Marcus Immanuvel Arockiasamy, Mechanicsville, VA (US); Sudheer Kumar Lagisetty, Glen Allen, VA (US); Satya Chigurupati, O'Fallon, MO (US); Akhil Srirama, Glen Allen, VA (US); Kameswari Srirama, Glen Allen, VA (US)

(72) Inventors: Padmanabaiah Srirama, Glen Allen, VA (US); Shankar Yalamanchili, Montgomerry, AL (US); Yashas Sai Chigurupati, O'Fallon, MO (US); Jesu Marcus Immanuvel Arockiasamy, Mechanicsville, VA (US); Sudheer Kumar Lagisetty, Glen Allen, VA (US); Satya Chigurupati, O'Fallon, MO (US); Akhil Srirama, Glen Allen, VA (US); Kameswari Srirama, Glen Allen, VA (US)

(73) Assignee: Fit Well Technologies Inc., Glen Allen, VA (US)

(21) Appl. No.: 19/231,505

(22) Filed: Jun. 8, 2025

Related U.S. Application Data
(60) Provisional application No. 63/697,552, filed on Sep. 22, 2024.

(51) Int. Cl. G06F 21/32; A61B 5/00; A61B 5/0205

(52) U.S. Cl. CPC G06F 21/32; A61B 5/0205; A61B 5/6802; A61B 5/7267; A61B 5/746

(57) ABSTRACT

A biometric-authenticated wearable system for remote monitoring, chronic disease management, elderly care, occupational therapy, and mental health rehabilitation is disclosed. The system uses fingerprint, facial, and voice recognition to securely associate physiological data with individual users. It monitors vital signs including heart rate, oxygen saturation, movement, and stress indicators, and transmits data to EMR/EHR systems in real time with privacy compliance. Machine learning modules assess disease risk, detect mobility decline, and perform sentiment-based analysis of speech and behavior. The system supports therapy compliance tracking across wellness programs and daily living tasks. The system may optionally employ large language models (LLMs) to enhance contextual understanding and sentiment interpretation from unstructured speech or text inputs. Real-time alerts are generated for health anomalies or non-compliance, enhancing clinical interventions. This integrated platform advances personalized and secure care through biometric authentication, predictive analytics, and seamless EMR/EHR integration.

### CROSS-REFERENCE TO RELATED APPLICATION

This application claims the benefit under 35 U.S.C. § 119(e) of U.S. Provisional Patent Application No. 63/697,552 , filed Sep. 22, 2024, for subject matter disclosed therein relating to biometric-authenticated health monitoring, wearable integration, and EMR/EHR-based real-time alert systems. The entire contents of the provisional application are incorporated herein by reference. Additional subject matter disclosed in the present application, including sentiment-based cognitive assessment and workflow logic described in Flows 6 and 7, constitutes new matter and is not part of the provisional application.

### FIELD OF THE INVENTION

This invention pertains to the fields of healthcare technology, biometric authentication, predictive AI-based health monitoring, and sentiment-based cognitive assessments. It focuses on the integration of wearable devices with electronic medical/health record (EMR/EHR) for applications in remote patient monitoring, chronic disease management, mental health rehabilitation, and occupational therapy tracking.

The invention builds upon prior technologies, including:
Issued Patent (U.S. Pat. No. 9,852,264; application Ser. No. 14/697,602), which introduces biometric authentication methodologies and wellness activities for secure data attribution to the correct user.
pending U.S. patent (application Ser. No. 18/784,779), which establishes supervised machine learning models for specific activity recognition based on the data from wearable devices.

Unlike the prior art, the present invention integrates these biometric and activity models into a unified system with real-time feedback loops, cross-domain therapy compliance, and context-aware sentiment analysis. It supports dynamic patient-specific insights across physical and cognitive domains, strengthening clinical decision-making.

The invention's modular workflows (see FIGS. 1-7) support a scalable architecture spanning biometric enrollment, condition-specific monitoring, and multi-actor interventions, adaptable across chronic disease, mental health, elderly care, and occupational therapy applications.

By leveraging these foundational technologies, the present invention extends the scope of healthcare monitoring through:
Real-time biometric authentication before storing health data into EMR/EHRs, ensuring accuracy and preventing data misattribution.
AI-driven predictive healthcare analytics, capable of detecting chronic disease risks, mobility decline, and mental health deviations.
Occupational therapy integration, tracking patient compliance with rehabilitation programs and therapy routines.
Sentiment-based mental health monitoring, using natural language processing (NLP) and behavioral data analysis for stress detection and emotional well-being tracking.

This unified system is designed for use in chronic disease management, elderly care, mental health rehabilitation, and occupational health tracking, ensuring real-time healthcare insights, corporate wellness programs, and compliance monitoring.

### BACKGROUND OF THE INVENTION

Management of fitness data has traditionally been performed manually, where participants record their exercise details such as times, dates, weights, repetitions, and sets for various activities. This data is typically entered into log books or paper media using writing instruments. Users later access these records to monitor their progress across different fitness parameters. However, analyzing such data manually is inefficient and time-consuming, as it involves consulting multiple records to draw meaningful conclusions about fitness progress. Traditionally this approach is used in many wellness centers for their members activity tracking purpose.

As an improvement over manual methods, some users have adopted digital solutions. These include manually typing fitness data into a computer and storing it as an electronic file. While this digital approach allows for better organization and easier access to data, the process of entering and maintaining the information remains labor-intensive and inconvenient. Another digital approach involves entering fitness data directly into portable electronic devices such as personal digital assistants (PDAs) at the time of exercise. Although this method facilitates real-time data entry, it requires users to carry electronic devices during their workouts, posing risks of loss, theft, and inconvenience.

The recent developments in wearable technology have introduced more sophisticated devices that can automatically capture a wide array of data, including motion, heart rate, and other biometric signals. These wearables are equipped with advanced sensors like accelerometers and gyroscopes, capable of recording detailed movement patterns. However, the challenge remains to effectively translate this raw data into actionable insights regarding specific physical activities, which is crucial for both casual users and fitness professionals. Some of the wearables provide tracking of wellness activity dashboard that includes the details like distance, calories burned, recorded blood pressure readings, duration of the activity and preliminary actionable insights based on the recorded activities.

Advances in healthcare technology have revolutionized the way patient data is collected, processed, and utilized. Wearable devices have emerged as essential tools for monitoring vital signs, tracking physical activity, and supporting chronic disease management. Over the past decade, wearable healthcare devices have become essential tools for remote patient monitoring (RPM), chronic disease management, and mental health rehabilitation. These devices have evolved from basic activity trackers to advanced biometric sensors that can track heart rate, oxygen saturation, movement patterns, and sleep quality.

The growing reliance on wearable technology for healthcare monitoring has led to innovations in chronic disease management, mental health rehabilitation, and remote patient care.

Additionally, traditional systems lack tools to assess cognitive and emotional well-being using natural, real-world interactions. The absence of sentiment-aware analysis limits the system's ability to proactively detect early signs of stress, depression, or cognitive decline. Most current solutions rely on subjective self-reporting or static questionnaires, which may not capture real-time emotional fluctuations.

With the emergence of natural language processing (NLP) and sentiment-aware artificial intelligence models—including optional large language models (LLMs)—it is now possible to analyze free-form speech or text inputs from users. These models can detect nuanced emotional states through linguistic patterns, tone, cadence, and vocabulary, enabling more accurate and timely mental health assessments. Despite these technological advances, existing systems have not yet integrated sentiment-based analysis into remote health monitoring workflows.

However, existing systems still exhibit significant limitations, particularly in ensuring the accuracy and security of patient data, seamless integration with Electronic Medical/Health Records (EMR/EHR), real-time intervention capabilities, and facilitating mental health rehabilitation, particularly when dealing with chronic disease patients, elderly care, rehabilitation therapy, and mental health monitoring.

### Limitations Of Current Systems

### 1. Insecure and Misattributed Data

Existing wearable systems often lack robust mechanisms for verifying the identity of the user. Without secure biometric authentication, data collected from wearable devices can be easily misattributed, leading to errors in diagnosis and treatment. This gap is addressed in the issued patent U.S. Pat. No. 9,852,264.
This issue is particularly critical in healthcare settings, where accurate and authenticated data is essential for clinical decision-making.
Example: A heart rate monitor could be worn by the wrong person, yet the data gets stored under a different patient's medical record.

### 2. Fragmented Systems and Poor Integration

Many wearable devices operate as standalone systems that do not integrate seamlessly with healthcare provider platforms or EMR/EHR systems. This fragmentation creates gaps in patient records, reducing the utility of wearables in long-term healthcare management.
Manual data transfer between wearable systems and EMR/EHRs is labor-intensive, prone to errors, and inefficient for both patients and providers.
Many hospitals and clinics lack seamless integration between patient wearables and electronic medical/health records.
Example: A diabetic patient may wear a glucose monitor, but healthcare providers do not receive real-time glucose data, leading to delayed medical interventions.

### 3. Challenges in Activity Recognition

While the pending patent (Ser. No. 18/784,779) advances activity recognition, its standalone application does not address real-time integration with clinical decision-making systems.

### 4. No Real-Time Monitoring for Chronic Health Patients

Patients suffering from diabetics, hypertension and cardiovascular deceases require continuous vital tracking due to the nature of these chronic conditions
The existing systems do not provide real-time alerts when sudden health
deviations occur.
Example: A hypertension patient experiencing a rapid spike in blood pressure will not receive an immediate alert, potentially leading to a critical emergency.

### 5. Challenges in Mental Health Rehabilitation

In mental health rehabilitation programs, verifying patient adherence to prescribed therapies—such as meditation, cognitive exercises, or physical rehabilitation—is often challenging. Current systems lack the ability to securely monitor and attribute these activities to specific patients.
The current mental health tracking systems rely on self-reported data, which is subjective and inconsistent.
Additionally, healthcare providers lack real-time insights into patient compliance, limiting their ability to adjust treatment plans proactively.
There is no mechanism to verify whether a patient has actually completed the prescribed therapy sessions, meditation exercises, or cognitive behavioral
Example: A patient may claim they completed a stress-reduction breathing exercise, but there is no biometric confirmation to verify compliance. therapy (CBT).

### 6. No Effective Occupational Therapy and Rehabilitation Tracking

Patients undergoing post-surgery rehabilitation, stroke recovery, or physical therapy must adhere to the prescribed exercises
Existing systems rely on adherence based on the patient reported adherence compliances
Example: A stroke patient prescribed daily mobility exercises may fail to complete them, but there is no wearable-based tracking system to notify caregivers.

### 7. Data Privacy Concerns

As healthcare data becomes increasingly digital, concerns about data privacy and security have grown. Wearable devices often fail to meet strict data encryption and privacy standards, exposing patient information to potential breaches.
Example: If a patient's wearable device is stolen, their medical data could be accessed and altered, compromising their electronic medical/health records.

### Opportunities for Improvement

The convergence of biometric authentication, wearable devices, and advanced data processing technologies presents an opportunity to overcome these challenges. By integrating these components into a unified system, the following goals can be achieved:
a) Accurate Data Attribution: Biometric authentication (U.S. Pat. No. 9,852,264) ensures that data collected by wearable devices is securely attributed to the correct patient, eliminating errors and misattribution.
b) Seamless EMR/EHR Integration: Automating the integration of wearable data with EMR/EHR systems reduces manual effort, minimizes errors, and provides healthcare providers with a comprehensive view of patient health.
c) Real-Time Monitoring and Alerts: Continuous monitoring of vital signs and activities enables healthcare providers to receive real-time alerts, allowing for timely interventions and improved patient outcomes.
d) Enhanced Mental Health Rehabilitation: The ability to track patient compliance with prescribed wellness activities ensures that mental health programs are more effective, with healthcare providers gaining real-time insights into progress.
e) Data Security and Privacy: Robust encryption and compliance with healthcare regulations like HIPAA ensure that patient data is securely transmitted and stored, protecting sensitive information.
f) Activity Recognition: Builds on the supervised learning models of Ser. No. 18/784,779, linking activity insights directly with healthcare systems for actionable clinical data for the health care professionals.
g) Predictive Healthcare Analytics: AI-driven models analyze historical patient data to detect early-stage health deterioration in diabetes, hypertension, mobility decline, and cognitive function, triggering proactive intervention alerts.
h) Sentiment based mental health tracking: The system incorporates sentiment-based mental health tracking that goes beyond traditional questionnaire-based evaluation. Using natural language processing (NLP) and optionally large language models (LLMs), the platform interprets free-form speech interactions, identifying indicators such as emotional tone, linguistic complexity, speech cadence, and sentiment shifts. This allows for early detection of emotional distress, cognitive anomalies, or behavioral deviations that might not be evident through physiological metrics alone. These insights are cross-referenced with biometric and behavioral data to generate comprehensive, real-time mental health assessments, enabling proactive interventions and personalized care planning.

Existing systems provide partial solutions but fail to address the comprehensive needs of modern healthcare:
a) Mental Health Monitoring Platforms: Current mental health rehabilitation tools focus primarily on subjective self-reported data rather than verified, real-time activity monitoring. This limits their reliability and effectiveness in clinical settings.
b) Chronic Disease Management Tools: Many systems designed for chronic disease management rely on patient-reported data or standalone devices. These approaches lack the real-time monitoring, biometric authentication, and EMR/EHR integration required for comprehensive care.

### Unmet Needs in Healthcare

There remains a critical need for a system that:
Combines biometric authentication with wearable devices to ensure secure and accurate data attribution.
Provides real-time monitoring of vitals and activity compliance, very important information for the health care provider.
Seamlessly integrates authenticated data into EMR/EHR systems to provide healthcare providers with a real-time, unified view of patient health along with the ICD10 codes.
Tracks compliance with mental health rehabilitation programs and chronic disease management protocols.
Ensures robust data security and privacy, addressing concerns about the misuse or breach of sensitive patient information.
Facilitates holistic healthcare solutions for mental health rehabilitation, chronic disease management, elderly care, and corporate wellness programs.

As such, it may be appreciated that there continues to be a need for a new and improved system and the present invention addresses these unmet needs by providing a Biometric-Authenticated Wearable System for Remote Monitoring, Chronic Disease Management, and Mental Health Rehabilitation. This system combines advanced biometric authentication technologies with wearable devices and EMR/EHR integration to offer a secure, real-time solution for modern healthcare challenges. It enables accurate data attribution, supports continuous patient monitoring, and enhances the effectiveness of mental health and chronic disease management programs, all while ensuring compliance with privacy standards. By building on the foundational patents, this invention represents a significant step forward in creating a unified, secure, and scalable healthcare monitoring system.

### SUMMARY OF THE INVENTION

The present invention builds upon prior innovations to create a unified system integrating biometric authentication, wearable device technologies, and Electronic Medical/Health Records (EMR/EHR). This invention addresses critical challenges in remote patient monitoring, mental health rehabilitation, and chronic disease management by combining:
1. Issued Patent (U.S. Pat. No. 9,852,264), which introduced advanced biometric authentication techniques for secure and accurate patient identification.
2. Pending patent (Ser. No. 18/784,779), which leverages supervised machine learning models to recognize physical activities from wearable device data.

The system utilizes biometric modalities—such as fingerprints, facial recognition, and voice recognition—to authenticate patient identity before recording and transmitting data. Wearable devices continuously monitor physiological parameters, including heart rate, blood oxygen levels, and movement patterns, ensuring that data collected is securely attributed to the authenticated individual, enabling the providers to have the critical actionable insights, patient outcomes and enhanced compliance with prescribed wellness activities.

For mental health rehabilitation, the system tracks compliance with prescribed activities, such as cognitive therapy exercises, meditation, or physical rehabilitation routines, providing healthcare providers with real-time insights into patient progress. In chronic disease management, the system supports monitoring of critical vitals like blood glucose levels and blood pressure, ensuring timely intervention and improved treatment outcomes. With seamless integration into EMR/EHR systems and robust data encryption, the invention addresses key challenges in healthcare, offering applications across multiple domains while adhering to data security standards like HIPAA.

This invention provides a comprehensive solution by seamlessly integrating these foundational technologies to ensure accurate data attribution, real-time monitoring, and secure health data management.

### Key Features of the Invention

a) User-Wearable Association and consent: Patients are linked to wearable devices through unique identifiers such as device serial numbers, ensuring precise data tracking. This association prevents cross-patient data errors and allows for seamless operation across multiple users. The user also provides consent to access the wearable record and updating of the user electronic medical record.
b) Biometric Authentication Integration: The system employs multiple biometric modalities such as fingerprint scanning, facial recognition, and voice recognition to authenticate the identity of patients (Expands the capabilities of U.S. Pat. No. 9,852,264). Authentication occurs at a linked electronic interface, ensuring that data captured is securely attributed to the correct patient. This layer of authentication eliminates risks of misattribution or data fraud, which are critical for healthcare applications where precise patient data is required for diagnosis and treatment.
c) User-Wearable device integration: Wearable devices monitor key physiological metrics, including heart rate, blood oxygen levels, blood pressure, respiratory rate, body temperature, and movement patterns. Advanced sensors, such as photoplethysmography (PPG) and accelerometers, enable continuous tracking, ensuring that even subtle changes in patient vitals are captured in real-time. Designed to integrate seamlessly with a wide range of wearable devices, the system supports new models and technologies as they emerge, ensuring long-term adaptability and relevance.
d) Enhanced Data Processing and Machine Learning: The invention utilizes advanced data processing techniques and intelligent analytics to continuously evaluate incoming physiological, behavioral, and contextual inputs from users. Central to this capability is a machine learning framework, which may include large language models (LLMs), that enables the system to interpret both structured and unstructured user inputs. These models analyze sensor outputs, text or voice-based responses, and activity patterns to generate meaningful insights and predictions. By integrating optional large language models LLMs, the system enhances its contextual understanding and decision-making ability, offering highly personalized health guidance and timely alerts for preventive care, tailored to each user's health profile and evolving conditions.
e) Associating the activity with the ICD10 code: The system will scan and assign the ICD10 activity code based on the detailed and granular activity performed by the user. Based on user consent, this activity data can be recorded into the electronic medical record for whole person health prospect.
f) Realtime monitoring and alerts: Continuously tracks vitals, movement, and therapy adherence, providing instant alerts to healthcare providers when abnormal health patterns or therapy non-compliance is detected, ensuring timely interventions.
g) Machine Learning for activity recognition: A supervised machine learning model processes data collected from wearable devices (Builds on the activity recognition models described in Ser. No. 18/784,779), identifying and classifying physical activities with high accuracy. The model improves over time, adapting to user-specific behaviors and wearable device features.
h) Scalability and Device Compatibility: The system is designed to integrate with a wide range of wearable devices, supporting future updates and new technologies as they emerge in the healthcare market.

### Mental Health Rehabilitation

Depending on prescribed care protocols, assessments may include both CMS-approved questionnaires and free-form user feedback, with analysis performed by sentiment-aware LLMs or structured ML models. The invention includes specialized features for tracking compliance with mental health programs. The system may also use sentiment-aware AI models—including optional LLMs—to interpret user mood and emotional shifts based on structured assessments like PHQ-9 and unstructured speech interactions. It monitors patient engagement in prescribed wellness activities, such as:
Cognitive therapy exercises, including brain training and stress management.
Physical rehabilitation routines, such as yoga or walking.
Meditation and mindfulness activities, measured through heart rate variability and movement tracking.

By securely linking authenticated data with EMR/EHR systems, the system provides healthcare providers with verified insights into patient progress, ensuring accurate tracking of prescribed therapies and enabling timely adjustments to treatment plans.

### Chronic Disease Management

The system is tailored for managing chronic diseases such as diabetes, hypertension, and cardiovascular conditions. analyzes patient responses and physiological patterns using predictive machine learning models, which may include large language models (LLMs), to identify early signs of deterioration and recommend proactive interventions, allowing healthcare providers to intervene promptly. Features include:
Blood glucose level tracking for diabetic patients.
Blood pressure and heart rate monitoring for hypertensive or cardiac patients.
Fall detection and mobility tracking for elderly patients, ensuring safety in assisted living or home-based care settings.

### Occupational Therapy and Elderly Care Management

Depending on prescribed care protocols, assessments may include both CMS-approved questionnaires and free-form user feedback, with analysis performed by sentiment-aware LLMs or structured ML models. The invention includes specialized features for tracking compliance with elderly care and occupational therapy programs. It monitors patient engagement in prescribed activities such as mobility exercises, cognitive tasks, sleep monitoring, and medication adherence. The application may prompt users to complete structured assessments, which can include CMS-approved tools such as the Activities of Daily Living (ADL) scale, Instrumental Activities of Daily Living (IADL) scale, and mood assessments like PHQ-9 or GAD-7. Large language models (LLMs) may optionally be used to analyze user feedback—both structured and free-form—to identify therapy-related concerns and enhance contextual understanding. Wellness routines, such as yoga and mindfulness exercises, are monitored through biometric and motion data. Post-surgical routines and home-based mobility compliance are continuously tracked to ensure adherence. By securely linking authenticated data with EMR/EHR systems, the platform provides caregivers and occupational therapists with verified, actionable insights, enabling timely interventions and adjustments to therapy plans. It monitors patient engagement in prescribed wellness activities, such as:
Mobility, Cognitive therapy exercises and vitals including stress management, sleep pattern, medication adherence.
Wellness routines, such as yoga or walking.
Meditation and mindfulness activities, measured through heart rate variability and movement tracking.
Structured assessments may include CMS-approved tools such as the ADL/IADL scales, with results analyzed using machine learning models and optionally LLMs to detect deviations in patient behavior and predict functional decline.
Home based mobility compliance.
Tracking post-surgical routines.

By securely linking authenticated data with EMR/EHR systems, the system provides healthcare providers and caregivers with verified insights into patient progress, ensuring accurate tracking of prescribed therapies and enabling timely adjustments to treatment plans.

### Applications

### 1. Remote Patient Monitoring (RPM):

Supports continuous tracking of vital signs, providing healthcare providers with real-time data for remote clinical decision-making.

### 2. Mental Health and Rehabilitation:

Facilitates monitoring and tracking of patient compliance with mental health therapies and rehabilitation programs, ensuring optimal care and treatment outcomes.

### 3. Elderly Care:

Tracks movement, detects falls, and monitors vitals, ensuring the safety of elderly patients in home or assisted living environments.

### 4. Corporate Wellness Programs:

Monitors employee stress, physical activity, and sleep quality, helping organizations promote better health among their workforce.

### 5. Occupational Therapy:

Tracks movement, detects falls, and monitors vitals, ensuring the safety of patients in home or assisted living environments.
Active Daily Living(ADL) activities and Independent Active Living activities(IADL) including medication management, meal preparation, communication, home-based mobility compliance and shopping.

### 6. Sentiment Analysis:

Tracks the changes in the patient tone, pattern, stress and vitals to alert the caregiver or to the professionals with the real-time intervention.
Helps in managing the stress with the cognitive exercises to stabilize the negative thought patterns and depressive symptoms.

### Advantages of the Invention

Improved Data Accuracy: Biometric authentication ensures that all data is securely attributed to the correct individual, eliminating errors in patient records. Eliminates the need for manual data entry and enhances the accuracy of activity tracking
Real-Time Interventions: Healthcare providers receive real-time data and alerts, enabling prompt responses to patient needs.
Enhanced Compliance Tracking: Tracks adherence to prescribed activities, helping providers ensure patients follow treatment plans.
Seamless EMR/EHR Integration: Automatically updates authenticated patient data into EMR/EHR systems for comprehensive healthcare documentation.
Flexibility and Scalability: Adapts to new wearable devices and models, ensuring continuous compatibility and growth.
Personalization: Customizes activity recognition based on individual user profiles and device-specific data.
Convenience monitoring and Efficiency: Provides a seamless and convenient method for diverse and comprehensive monitoring including mental health, chronic and elderly care, physical activities without the need for carrying additional devices or manual inputs.
Emotion-aware interventions: Alerts triggered by changes in speech tone, stress levels, or behavioral cues allow early mental health support
Advanced Contextual Interpretation: Optional large language models (LLMs) provide deeper analysis of user-reported feedback and free-form input, enhancing detection of subtle behavioral or emotional deviations.

By integrating biometric authentication, wearable technology, and EMR/EHR systems, this invention addresses critical challenges in healthcare, offering a secure, real-time, and accurate solution for remote monitoring, chronic disease management, and mental health rehabilitation. This invention represents a significant advancement in the field of remote patient monitoring and associated healthcare area.

### BRIEF DESCRIPTION OF THE DRAWINGS

To further clarify various aspects of some example embodiments of the present invention, a more particular description of the invention will be rendered by reference to specific embodiments thereof which are illustrated in the appended drawing. It is appreciated that the drawing depicts only illustrated embodiments of the invention and are therefore not to be considered limiting of its scope. The invention will be described and explained with additional specificity and detail through the use of the accompanying drawing in which:

FIG. 1 illustrates a system-level architecture flow diagram illustrating the integration of biometric authentication, wearable device data collection, and secure transmission to electronic medical/health records (EMR/EHR) for real-time remote patient monitoring.

FIG. 2 illustrates a workflow diagram depicting the biometric authentication process and patient attribution mechanism, showing how biometric data is captured, encoded, and used to verify user identity prior to health data integration with EMR/EHR systems.

FIG. 3 illustrates a process flow diagram for managing chronic disease monitoring, illustrating the scheduling of wellness activities, continuous vitals tracking, and predictive analytics, including optional use of large language models (LLMs), for early intervention and context-aware recommendations.

FIG. 4 illustrates a flowchart illustrating the monitoring and management of elderly care, including fall detection, cognitive health assessment using structured and optionally CMS-approved tools, therapy adherence tracking, and proactive alert notification workflows enhanced by predictive analytics and LLMs.

FIG. 5 illustrates a workflow for mental health rehabilitation using biometric-authenticated wearables, sentiment-based analysis using voice input and emotional indicators, with predictive modeling supported by machine learning and optionally large language models (LLMs), along with activity tracking and therapy compliance validation.

FIG. 6 illustrates a flow diagram for sentiment analysis-based health monitoring, showing how speech, behavior, and emotional indicators are interpreted using sentiment-aware machine learning models, which may include large language models (LLMs), to detect deviations and trigger real-time alerts.

FIG. 7 illustrates a workflow for occupational therapy services, illustrating biometric verification, movement tracking, and structured assessments including CMS-approved tools such as ADL/IADL scales and mood ratings, with predictive analytics supported by LLMs, integrated into EMR/EHR systems for occupational therapy progress reporting.

### DETAIL DESCRIPTION OF THE INVENTION

### Introduction

Various aspects of the illustrative embodiments will be described using terms commonly employed by those skilled in the art to convey the substance of their work. The present invention may be practiced with only some of the described aspects. Specific numbers, materials, and configurations are set forth to provide a thorough understanding of the illustrative embodiments. However, it will be apparent to one skilled in the art that the present invention may be practiced without these specific details.

The present invention integrates biometric authentication, wearable devices, and machine learning models into a unified system for remote patient monitoring, mental health rehabilitation, corporate wellness programs, elderly care, and chronic disease management. It builds upon:
1. Issued Patent U.S. Pat. No. 9,852,264, which introduces biometric authentication methodologies for secure and accurate data attribution.
2. Pending patent application Ser. No. 18/784,779, Which Establishes

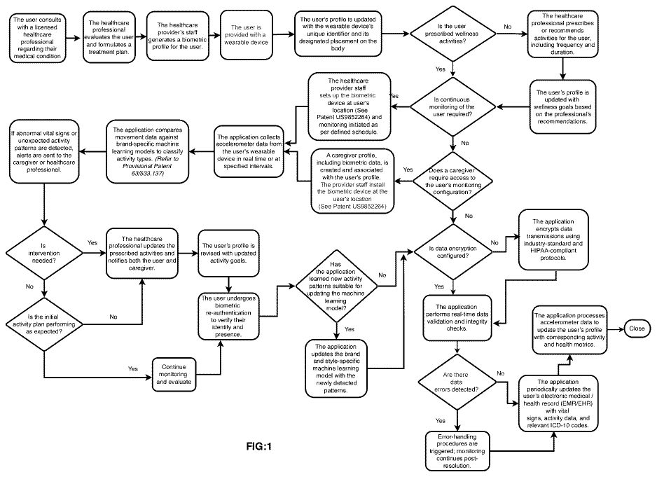

supervised machine learning models for activity recognition. This invention addresses critical gaps in healthcare technology by ensuring accurate patient data attribution, real-time monitoring, and seamless integration with EMR/EHR systems, while adhering to strict privacy standards like HIPAA. The invention is described in reference to FIGS. 1-7, with corresponding functional flows supported in Section A and Section B claims.

### Section1: System Architecture and Workflow Overview (FIG. 1)

FIG. 1 illustrates a system-level architecture flow for a biometric-authenticated wearable device system, configured to support real-time remote monitoring of patients. The process initiates with the user consulting a licensed healthcare professional regarding a medical condition. Upon evaluation, the healthcare professional formulates a personalized treatment plan that may include activity recommendations and monitoring requirements.

The healthcare provider's personnel create a biometric profile for the user, capturing physiological identifiers. This biometric data is not stored locally; instead, it is converted into encrypted biometric codings and securely transmitted to a centralized server for storage and future authentication.

The user is then provisioned with a wearable monitoring device. The wearable device's unique identifier and its anatomical placement are recorded in the user's profile. The system processes data collected from the device's sensors—such as accelerometers and vital sign monitors—and updates the user's profile with real-time activity and health metrics. In cases where anomalies or data inconsistencies are detected, the system performs automatic recalibration and initiates corrective measures.

If caregiver access is needed, a caregiver profile is generated and linked to the user's account. This includes secure biometric identification for the caregiver, as described in U.S. Pat. No. 9,852,264. Where continuous monitoring is warranted, provider personnel install biometric monitoring equipment at the user's location.

The application collects accelerometer data in real time or at specified intervals and interprets it using brand-specific machine learning models to classify user activity. If abnormal patterns or critical vitals are detected, alerts are triggered for caregiver or healthcare professional intervention.

All data transmissions are encrypted using industry-standard, HIPAA-compliant protocols. User presence is continuously verified via biometric re-authentication to ensure secure attribution of data to the correct individual. The application periodically updates the user's electronic medical/health record (EMR/EHR) with validated activity metrics, vital signs, and ICD-10 codes.

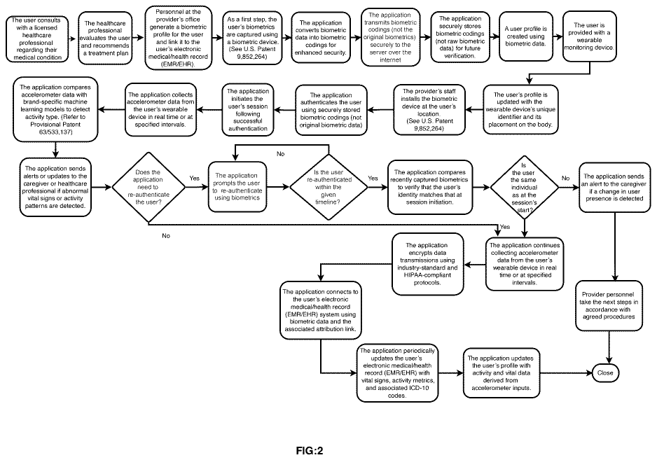

### Section2: Biometric Authentication and Patient Attribution (FIG. 2)

FIG. 2 illustrates a workflow diagram representing the biometric authentication and patient attribution process within the biometric-authenticated wearable device system. This workflow supports secure user identity verification, ensures that health data is accurately attributed, and integrates with electronic medical/health records (EMR/EHR) in a privacy-compliant manner.

The process begins when a user consults a licensed healthcare professional to discuss a medical condition. Following evaluation, the healthcare professional formulates a treatment plan and initiates the enrollment process.

Provider personnel create a biometric profile for the user using a biometric capture device. At the point of capture, the system does not store raw biometric data locally. Instead, the biometric input—such as a facial scan, fingerprint, or voice sample—is immediately processed into a secure, encrypted biometric coding. This coding is transmitted over a secure internet connection to a centralized server, where it is stored in encrypted form for future authentication use. At no time is the original biometric data retained in local memory, ensuring data privacy and reducing exposure risk. At any point of time, the stored coding can be decrypted back to the original raw biometric format.

The biometric coded profile is then associated with the user's electronic medical or health record (EMR/EHR), forming a secure attribution link between the individual and their medical data. Simultaneously, a user profile is generated within the system that connects the biometric identity to the user's health records and device credentials.

The user is issued a wearable monitoring device that continuously collects physiological data such as heart rate, motion, and temperature. The system updates the user's profile with the wearable's unique identifier and anatomical placement details. If necessary, provider staff install a biometric device at the user's home, in accordance with procedures outlined in U.S. Pat. No. 9,852,264.

When a user accesses the system, biometric input is captured and converted again into encrypted codings, which are compared against the previously stored version on the server. If the codings match, the user is authenticated, and a session is initiated. This biometric authentication confirms user presence and ensures that health data collected during the session is attributed correctly.

The system is capable of prompting for re-authentication during an active session if continuous identity confirmation is required or if it detects a change in user interaction. The re-authentication process involves capturing new biometric input and verifying that it matches the session-initiating identity. If re-authentication fails, or if the user's identity does not match, the system generates an alert that is sent to caregivers or healthcare personnel, and escalation protocols are triggered based on predefined rules.

During authenticated sessions, the wearable device transmits real-time accelerometer data and vital signs to the application. These data streams are analyzed using brand-specific machine learning models to detect and classify user activity types, as described in Provisional Patent Application No. 63/533,137. The system identifies anomalies such as unusual movement or deviations in vitals.

If abnormal patterns are detected—such as irregular heart rates, fall-like motion, or missed medication cues—the system sends alerts to designated caregivers or clinical staff for timely intervention.

All transmitted data is encrypted using HIPAA-compliant, industry-standard protocols. Before storing data into the EMR/EHR system, the system performs biometric verification to ensure accurate identity attribution. This ensures that only health data associated with an authenticated individual is recorded, preserving both the security and medical validity of the information.

This biometric workflow supports a secure, privacy-conscious method for verifying patient identity, continuously attributing health data, and enabling safe medical decision-making in real time.

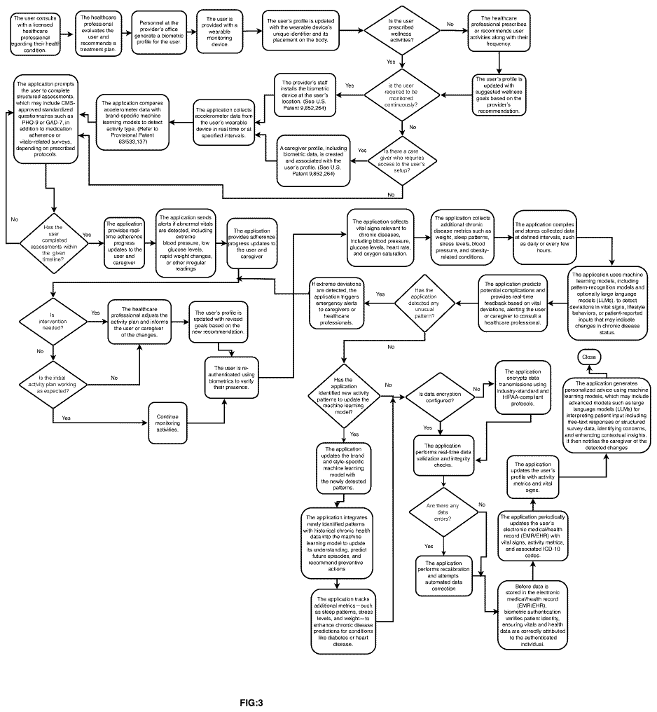

### Section 3: Chronic Disease Monitoring and Management (FIG. 3)

FIG. 3 illustrates a workflow for monitoring and managing chronic disease conditions using a biometric-authenticated wearable device system. This process enables secure, real-time tracking of chronic health indicators, medication adherence, user activity, and machine learning-based predictive analytics tailored to individual patient profiles.

The process begins with the user consulting a licensed healthcare professional to discuss their chronic health condition. Based on the clinical evaluation, the healthcare professional recommends a treatment plan which may include lifestyle adjustments, medication adherence monitoring, and continuous or periodic tracking of key health metrics.

Provider personnel then generate a biometric profile for the user. Upon biometric capture, no raw biometric data is stored locally. Instead, biometric inputs are securely converted into encrypted biometric codings and transmitted to a central server for future authentication use. The user is provided with a wearable monitoring device, and the system updates the user profile with the device's unique identifier and its designated placement on the body.

The application initiates tracking by collecting data from the wearable, including activity metrics and vital signs relevant to chronic disease conditions—such as blood pressure, glucose levels, oxygen saturation, and heart rate. Collected data is used to update the user profile and may be subject to recalibration and automated correction routines to ensure accuracy.

If a caregiver is involved in the user's health plan, a caregiver profile is generated and associated with the user. This caregiver profile includes secure biometric attributes and is used to grant authorized access, as described in U.S. Pat. No. 9,852,264.

Where continuous monitoring is required, biometric devices are installed at the user's location by provider staff. The healthcare professional may also prescribe wellness activities with defined frequency and duration, which are then incorporated into the user's monitoring schedule and profile.

The system collects accelerometer data either in real time or at user-defined intervals and compares this data against brand-specific machine learning models to classify user activity types, as disclosed in Provisional Ser. No. 63/533,137. These models help identify adherence to prescribed activities and detect potential concerns, such as inactivity or irregular behavior.

If data encryption is not already configured, the application activates secure transmission protocols using HIPAA-compliant, industry-standard encryption methods. The system also ensures that any sensitive data updates—such as health readings or behavioral trends—are securely handled only after biometric re-authentication confirms the user's presence. Biometric re-authentication may be prompted periodically or in response to a change in user presence. If re-authentication fails or is not completed within a specified timeline, alerts are generated for caregiver or healthcare provider review. Upon successful re-authentication, the application resumes continuous monitoring and proceeds with validated updates to the user's EMR/EHR.

The system performs real-time data validation and integrity checks before updating the electronic medical/health record with vitals, activity metrics, and relevant ICD-10 codes. If data errors are detected, the system attempts correction protocols or flags the anomaly for clinical review.

Users are prompted to complete medication adherence assessments or vitals-related surveys at designated intervals. If the user completes these assessments within the assigned window, adherence progress is tracked and reported to both the user and caregiver. If non-compliance is detected, follow-up alerts are issued.

Data compiled over time is stored at predefined intervals, such as hourly or daily, and includes key chronic disease indicators. The application applies machine learning to detect deviations from baseline patterns that may signal a deterioration in health status. If such anomalies are found, the system generates personalized recommendations and informs caregivers accordingly.

Additionally, the system predicts potential complications based on observed changes in vitals or behavior. Additionally, optional large language models (LLMs) may be employed to analyze unstructured user input or conversational assessments. This enables context-aware detection of behavioral changes or emotional fatigue that may correlate with chronic disease progression or treatment adherence. It provides real-time feedback and alerts the user or caregiver to consult a healthcare provider when needed. Extreme deviations automatically trigger emergency alerts to designated caregivers or medical personnel.

The application further tracks additional chronic disease metrics—including sleep patterns, stress levels, weight changes, and obesity-related conditions—which are used to enhance predictive accuracy for diseases such as diabetes, cardiovascular illness, and hypertension.

Finally, before any health data is recorded in the EMR/EHR, the system conducts biometric authentication to ensure accurate attribution to the correct individual. This guarantees that all stored information is tied to a verified identity, supporting clinical decision-making and maintaining data integrity across the healthcare lifecycle.

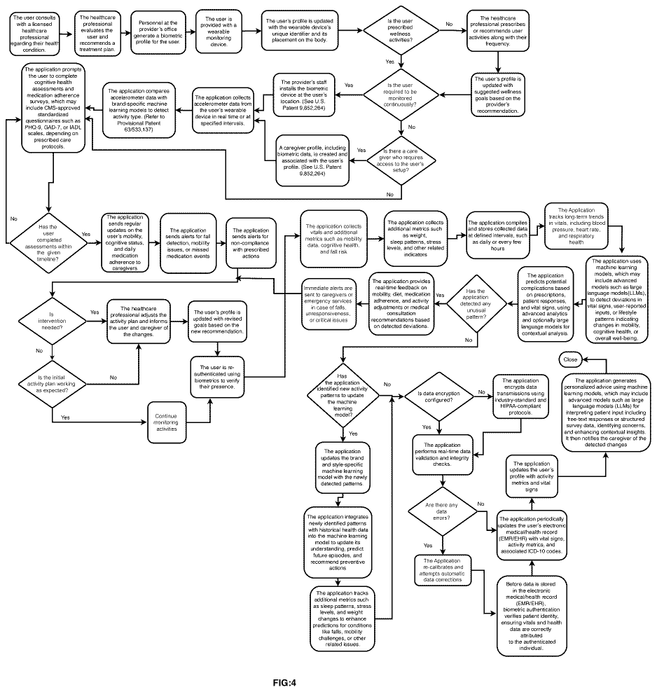

### Section 4: Elderly Care Monitoring and Management (fig. 4)

FIG. 4 illustrates a detailed workflow for monitoring and managing elderly care using a biometric-authenticated wearable device system. The process encompasses biometric identity verification, secure activity tracking, machine learning-based anomaly detection, and caregiver alert systems—designed to support the unique health and safety needs of elderly individuals.

The workflow begins with a healthcare consultation, during which the licensed professional evaluates the user and formulates a treatment plan tailored to the user's condition. This plan may include activity recommendations, continuous monitoring directives, and cognitive health assessments.

Provider personnel then create a biometric profile for the user. At the point of biometric capture, raw biometric data is not stored locally. Instead, the input is immediately converted into encrypted biometric codings, which are securely transmitted to a central server. These codings are used for future authentication, ensuring that only validated individuals can interact with the system. The biometric profile is linked to the user's electronic medical or health record (EMR/EHR) for secure attribution.

A wearable monitoring device is issued to the user. The system records the unique identifier of the device and its designated body placement. The application begins collecting physiological and activity-related data from the device, which may include heart rate, mobility patterns, and vital signs. The system updates the user's profile accordingly and applies recalibration routines and automatic data correction mechanisms when irregularities are detected.

If caregiver involvement is required, a biometric-authenticated caregiver profile is created and linked to the user's account, as outlined in U.S. Pat. No. 9,852,264. Where continuous monitoring is part of the care plan, provider staff install the necessary biometric hardware at the user's location.

The system records prescribed wellness activities and updates the user's profile with corresponding goals and expected frequency. Real-time or scheduled accelerometer data is analyzed using brand-specific machine learning models to detect and classify user activities, referencing methodologies detailed in Provisional Ser. No. 63/533,137.

The system checks if encryption is configured. If not, it activates secure, HIPAA-compliant data transmission protocols. Biometric re-authentication is conducted before any sensitive data operation to confirm the user's presence and ensure the integrity of data attribution. If re-authentication fails, or the presence of the user cannot be confirmed, alerts are issued to caregivers or healthcare providers.

The application performs real-time validation and integrity checks on incoming data. If errors are found, the system attempts automated correction. Validated data, including vital signs, cognitive status, and activity metrics, is periodically uploaded to the EMR/EHR system along with applicable ICD-10 codes.

Cognitive health assessments and medication adherence surveys are periodically prompted. If users fail to complete these within the prescribed timeline, alerts for non-compliance are triggered. When assessments are completed on time, the system logs progress and updates both the user and caregiver.

The system aggregates additional metrics such as mobility data, fall risk, and cognitive performance. It compiles this information at predefined intervals (e.g., daily or every few hours) and tracks long-term trends in blood pressure, heart rate, and respiratory health.

Machine learning algorithms analyze deviations in lifestyle and vitals to detect early signs of decline in mobility, cognitive function, or general well-being. If new activity or behavior patterns are detected, the models are updated using historical data to improve prediction accuracy and tailor preventive care recommendations. If extreme deviations are identified, immediate alerts are dispatched to caregivers or emergency services. Additionally, the system may optionally incorporate large language models (LLMs) to analyze spoken or text-based assessments, enabling early detection of subtle cognitive shifts, mood changes, or social withdrawal often associated with elderly cognitive decline.

The application also tracks ancillary health indicators, such as sleep quality, stress levels, and weight changes, to enhance prediction models for elderly care issues, including fall risk and mobility deterioration. In some cases, it provides personalized advice based on prescription profiles and observed physiological trends, prompting the user or caregiver to consult a healthcare professional.

Prior to final data submission to the EMR/EHR, biometric re-authentication is conducted to ensure correct patient attribution. This security measure guarantees that health data is only associated with a verified individual, preserving medical accuracy and protecting patient privacy.

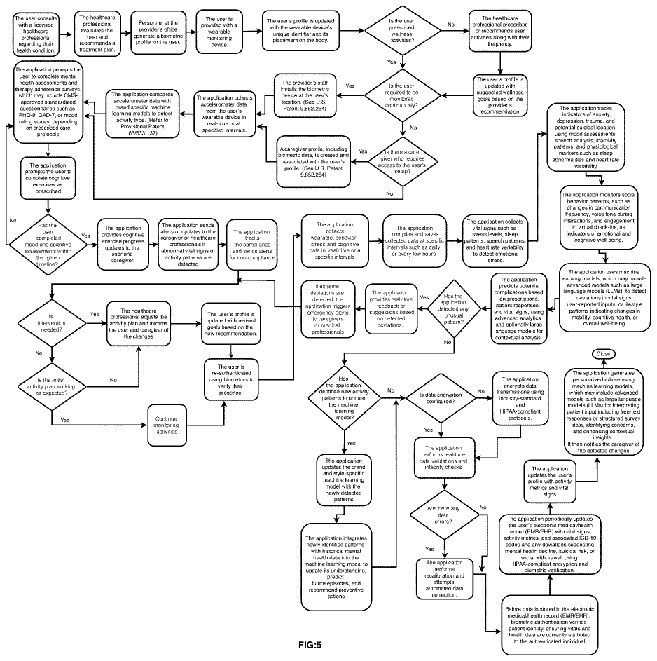

### Section5: Mental Health Rehabilitation Monitoring and Management (FIG. 5)

FIG. 5 Illustrates a workflow for mental health rehabilitation utilizing a biometric-authenticated wearable device system. This system enables continuous monitoring and intelligent analysis of user behavior, physiological trends, emotional indicators, and cognitive compliance, supporting early detection and intervention for mental health conditions such as depression, anxiety, trauma, and social withdrawal.

The process begins with the user consulting a licensed healthcare professional regarding their mental health condition. Based on this consultation, the healthcare professional evaluates the user and formulates a treatment plan which may include wellness activities, cognitive exercises, and continuous behavioral tracking.

Provider personnel create a biometric profile for the user using secure biometric capture mechanisms. No raw biometric data is stored locally; instead, captured data is immediately converted into encrypted biometric codings and transmitted to a centralized server. These codings are used solely for authentication and attribution, maintaining privacy and data integrity. The biometric profile is linked with the user's electronic medical or health record (EMR/EHR).

The user is provided with a wearable monitoring device. The user profile is updated with the device's unique identifier and its anatomical placement. The application collects data from the device, including physiological signals and activity metrics, which are used to update the user's profile. Automated calibration and data correction procedures are applied where necessary to maintain accuracy.

If caregiver access is needed, a secure caregiver profile is created and associated with the user's account, using biometric credentials as disclosed in U.S. Pat. No. 9,852,264. If continuous monitoring is required, provider staff install biometric devices at the user's residence, enabling persistent data collection and system interaction.

The system logs prescribed mental health and wellness activities, along with target frequency and duration. Real-time accelerometer data is collected and analyzed using brand-specific machine learning models to classify user activity, as described in Provisional Ser. No. 63/533,137.

The application checks whether encryption is configured; if not, HIPAA-compliant encryption protocols are activated to secure all data transmissions. Prior to sensitive operations, the user undergoes biometric re-authentication to verify their presence. If re-authentication fails or the user identity cannot be validated, access is restricted, and alerts may be sent to caregivers.

The application collects and processes behavioral and cognitive health data in real time or at specified intervals. These models may optionally include large language models (LLMs), which analyze linguistic and contextual cues from user responses to improve emotional state detection and therapy personalization. This enables context-aware detection of behavioral changes, emotional withdrawal, or therapy disengagement that may indicate mental health deterioration. If anomalies are detected in activity or vital signs—such as indicators of stress, inactivity, or speech changes—alerts are issued to caregivers or healthcare providers. Based on the healthcare provider's response, activity goals may be revised, and the user profile is updated accordingly.

The application periodically prompts the user to complete mood assessments and prescribed cognitive exercises. It tracks whether these are completed within the expected timeframe and sends alerts in cases of non-compliance. Completion results and adherence progress are shared with both the user and caregivers.

The system aggregates and stores data at defined intervals, including sleep patterns, speech data, heart rate variability, and stress levels. Machine learning algorithms assess this data to detect patterns indicating mental health changes, such as anxiety, depression, suicidal ideation, or cognitive decline.

The application generates real-time feedback and personalized suggestions based on detected deviations. If new behavioral patterns are identified, these are integrated into the system's machine learning models to enhance predictive accuracy and generate early warnings. Extreme deviations automatically trigger emergency alerts to caregivers or medical professionals.

Additionally, the system monitors social interaction metrics, such as communication frequency, voice tone, and participation in virtual check-ins. These are analyzed as indicators of emotional and cognitive well-being.

Before any data is committed to the EMR/EHR system, the user's biometric identity is verified. This ensures that all recorded health data is attributed to the authenticated individual, maintaining clinical integrity and legal compliance.

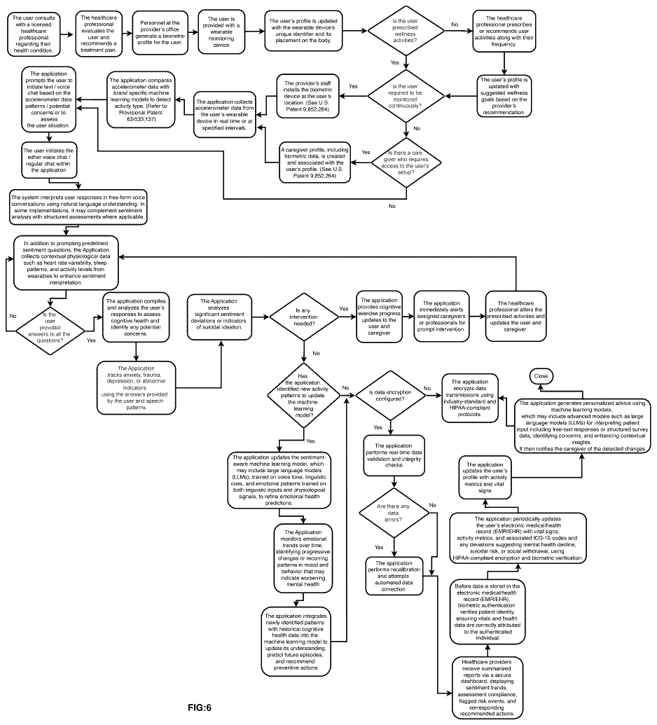

### Section 6: Sentiment Analysis-Based Health Monitoring (FIG. 6)

FIG. 6 illustrates a workflow for sentiment analysis-based health monitoring using a biometric-authenticated wearable device system. This system integrates natural language processing, biometric identity verification, physiological sensing, and machine learning to detect emotional deviations, mental health risks, and cognitive decline, providing real-time support and intervention in a privacy-secure manner.

The process begins with a user consulting a licensed healthcare professional regarding their condition. Following evaluation, the professional develops a treatment plan, which may include sentiment tracking, emotional health assessments, and wellness activities.

Personnel at the provider's office create a biometric profile for the user. Raw biometric data is not stored locally; instead, the biometric inputs are immediately transformed into encrypted biometric codings. These codings are securely transmitted to and stored on a central server, where they are used solely for future identity authentication.

The user is then provisioned with a wearable monitoring device. The user's profile is updated with the device's unique identifier and its physical placement. The application collects data from the wearable and updates the profile with vital signs and activity metrics. Recalibration and automated data correction routines are applied where needed.

If a caregiver requires access, a secure, biometric-linked caregiver profile is created and associated with the user, following the approach disclosed in U.S. Pat. No. 9,852,264. If continuous sentiment monitoring is required, biometric devices may be installed at the user's location.

Wellness and mental health activities are prescribed and recorded. The application collects accelerometer and physiological data in real time or at set intervals, using brand-specific machine learning models to classify activities, as referenced in Provisional Patent Application No. 63/533,137. The system also assesses user responses to prompted sentiment questions or interactions initiated by behavioral patterns detected through wearable inputs.

The application may prompt users to engage in voice or text-based chats within the system interface to evaluate their emotional state. It tracks indicators such as speech tone, answer content, communication frequency, and other social interaction cues. These are analyzed in combination with contextual physiological data—such as heart rate variability, sleep patterns, and activity levels—to refine sentiment interpretation. Optional use of large language models (LLMs) enables deeper contextual interpretation of emotional tone, cognitive state, and linguistic deviation patterns, supplementing traditional sentiment scoring. This enables context-aware detection of behavioral changes or emotional fatigue that may correlate with chronic disease progression or treatment adherence.

If encryption is not configured, the application initiates HIPAA-compliant encryption protocols. Before storing any data or initiating sensitive processes, biometric re-authentication is performed to confirm user presence. If identity verification fails, alerts are dispatched to caregivers or clinical staff.

All collected data is subjected to real-time validation and integrity checks. If anomalies are detected, the system performs automatic corrections or flags the data for review. The system aggregates the collected physiological and behavioral data, storing it at regular intervals such as daily or every few hours.

The application tracks sentiment deviations including signs of depression, anxiety, trauma, or suicidal ideation. If a deviation or risk indicator is detected, alerts are generated. The healthcare provider may adjust prescribed activities and communicate changes to the user and caregiver. These updates are reflected in the user's profile.

Personalized advice is generated in response to detected emotional shifts. If newly observed sentiment patterns are identified, they are integrated into the sentiment-aware machine learning model, which incorporates linguistic and physiological signals. This updated model supports predictive insight and preventive care strategies.

Healthcare providers receive structured summaries through a secure dashboard, which includes sentiment trends, assessment compliance, flagged risks, and recommended interventions. Before any data is entered into the EMR/EHR, biometric authentication ensures that all health data is properly attributed to the authenticated user.

This sentiment-driven monitoring framework allows for dynamic, individualized emotional health evaluation while maintaining strict identity and data security standards.

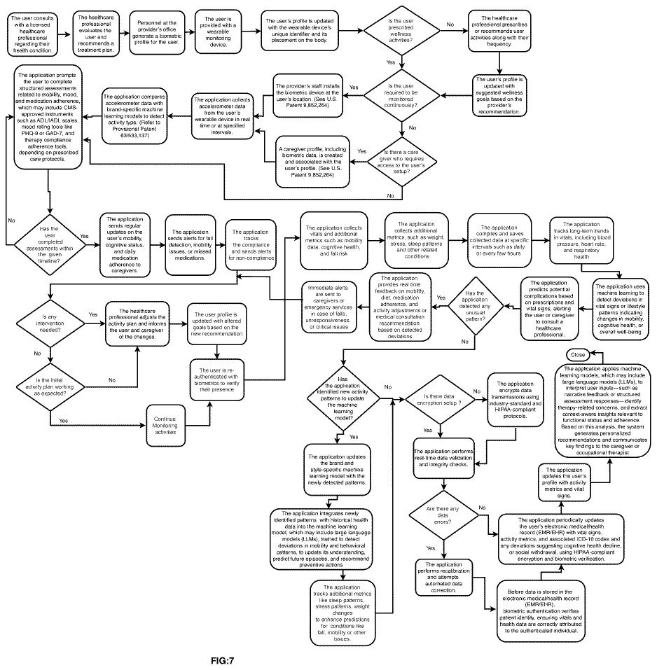

### Section7: Occupational Therapist Patients Remote Monitoring and Management (FIG. 7)

FIG. 7 illustrates a workflow for occupational therapy management utilizing a biometric-authenticated wearable device system. This system is configured to monitor activities of daily living (ADL), instrumental activities of daily living (IADL), cognitive and physical therapy compliance, and overall functional status. The workflow supports personalized rehabilitation through real-time feedback, anomaly detection, secure data attribution, and adaptive machine learning.

The process begins with a user consulting a licensed healthcare professional regarding limitations or conditions affecting daily function. Following the evaluation, the professional formulates an occupational therapy treatment plan, which may include movement therapy, ADL/IADL training, cognitive exercises, and mobility tracking.

A biometric profile is generated for the user by provider personnel. Raw biometric inputs are securely converted into encrypted biometric codings and transmitted to a central server. These codings are never stored locally and are used solely for identity authentication. The user's biometric profile is linked to their electronic medical or health record (EMR/EHR), ensuring

secure and accurate attribution of all subsequent health data. The user is issued a wearable monitoring device, and their profile is updated with the device's unique identifier and anatomical placement. The system begins collecting physiological and motion data—such as step count, range of motion, heart rate, and postural changes—and updates the profile accordingly. Automated recalibration and data correction procedures are applied to ensure data accuracy.

If a caregiver requires access, a biometric-authenticated caregiver profile is created and linked to the user account, following the framework disclosed in U.S. Pat. No. 9,852,264. When continuous monitoring is prescribed, biometric monitoring equipment is installed at the user's location by provider staff.

Prescribed therapy goals include daily movement targets, ADL/IADL checklists, cognitive engagement activities, and medication adherence tasks. The application tracks user compliance with these goals using real-time accelerometer data and brand-specific machine learning models, as referenced in Provisional Ser. No. 63/533,137.

If encryption is not configured, the system activates HIPAA-compliant secure transmission protocols. Biometric re-authentication is conducted prior to critical operations to verify user identity. If re-authentication fails, access is blocked and alerts are issued to the care team.

The application prompts the user to complete routine assessments for ADL proficiency, mobility level, cognitive sharpness, and medication adherence. Results are analyzed for compliance, and alerts are generated if required tasks are not completed within the prescribed window. These updates are logged in the user profile and shared with caregivers. Optionally, large language models (LLMs) may be used to interpret user interactions, such as spoken feedback or journal-style inputs, helping identify motivational issues, therapy fatigue, or cognitive stagnation that may require plan revision. This enables context-aware detection of behavioral changes or emotional fatigue that may correlate with chronic disease progression or treatment adherence.

Collected data includes vital signs, postural shifts, fine motor activity, gait stability, and reaction time. The system compiles this data at defined intervals (e.g., hourly, daily), monitoring long-term trends in mobility, cognitive response, and functional engagement. Real-time validation ensures data integrity prior to integration with the EMR/EHR.

The application applies machine learning to detect deviations in mobility patterns, changes in ADL/IADL capabilities, and indicators of therapy regression. When new behavioral or movement patterns are detected, they are incorporated into user-specific predictive models. Personalized recommendations—such as modifying exercise routines, adjusting movement goals, or scheduling clinical check-ins—are generated and delivered to both the user and the caregiver.

Immediate alerts are triggered for critical situations such as sudden mobility loss, cognitive disorientation, or missed medications. The system tracks auxiliary health indicators—such as sleep patterns, stress levels, and weight changes—to enhance prediction models related to therapy progress, fall risk, and rehabilitation trajectory.

If intervention is deemed necessary, the healthcare professional updates the therapy plan, and the user's profile is revised accordingly. The system provides real-time feedback on therapy participation, cognitive function, and ADL/IADL performance. Regular updates summarizing compliance and trends are transmitted to caregivers and healthcare professionals.

Before any data is committed to the EMR/EHR, biometric verification is performed to confirm user identity. This ensures that all clinical records are securely and accurately attributed to the authenticated individual, upholding the medical validity of the rehabilitation process.

1. A biometric-authenticated wearable healthcare monitoring system comprising:
(a) at least one wearable device configured to continuously capture physiological data from a user, including at least one of: heart rate, oxygen saturation, movement patterns, blood pressure, or respiratory rate;
(b) a biometric authentication module configured to verify the identity of the user using one or more biometric modalities selected from the group consisting of fingerprint recognition, facial recognition, and voice recognition;
(c) a processing module configured to associate the physiological data with the user's identity, as confirmed by successful biometric verification; an integration interface configured to securely transmit the authenticated and attributed physiological data into an electronic medical or health record (EMR/EHR) system in substantially real time or at predefined intervals;
(d) a monitoring module configured to generate alerts in response to detection of abnormal health conditions or deviations from prescribed activity thresholds.

2. The system of claim 1, wherein the wearable device is further configured to detect user compliance with prescribed therapy routines including cognitive exercises, physical rehabilitation, or meditation.

3. The system of claim 1, wherein the biometric authentication occurs prior to each data transmission to ensure secure attribution.

4. The system of claim 1, wherein the processing module comprises a machine learning model trained to classify physical activities performed by the user based on sensor data from the wearable device.

5. The system of claim 1, further comprising a sentiment analysis module configured to evaluate speech-based inputs from the user, including tone, pace, and linguistic patterns.

6. The system of claim 5, wherein the sentiment analysis module is further configured to determine the user's emotional state based on the evaluated speech inputs and contextual biometric indicators.

7. The system of claim 1, wherein the integration interface is further configured to assign an ICD-10 activity code to the captured data prior to updating the EMR/EHR system.

8. The system of claim 1, wherein the wearable device is further configured to monitor compliance with activities of daily living (ADLs) and instrumental activities of daily living (IADLs).
section B—Claims Based on Non-Provisional Enhancements (LLM/NLP Capabilities) The following claims pertain to enhancements introduced after the provisional filing and may incorporate optional large language model (LLM) functionality for extended contextual interpretation and sentiment analysis.

9. A method for sentiment-based cognitive assessment and real-time alert generation in a wearable healthcare monitoring system, the method comprising:
(a) receiving voice input and biometric sensor data from a user via a wearable device;
(b) analyzing the voice input using natural language processing (NLP) to detect sentiment indicators including tone, word choice, and speech rate;
(c) correlating the sentiment indicators with physiological stress signals to assess cognitive and emotional health status;
(d) and generating real-time alerts to healthcare providers when cognitive anomalies or emotional distress are detected.

10. The method of claim 9, further comprising updating the user's electronic medical or health record (EMR/EHR) with the sentiment analysis data and corresponding biometric indicators.

11. The method of claim 9, wherein the biometric sensor data includes at least one of: heart rate variability, respiratory rate, or electrodermal activity.

12. The method of claim 9, wherein the real-time alerts are transmitted via secure messaging channels or integrated clinical dashboards.

13. The method of claim 9, wherein the natural language processing is optionally performed using a large language model (LLM) trained on clinical or psychological data to enhance the accuracy of sentiment and behavioral health analysis.

14. The system of claim 1, wherein chronic disease-related assessments are optionally enhanced using a large language model (LLM) that interprets unstructured user inputs to detect emotional fatigue, behavioral change, or deviation from treatment adherence.

15. The system of claim 1, wherein the elderly care module optionally incorporates a large language model (LLM) to detect early cognitive decline or social disengagement from spoken or written inputs.

16. The system of claim 5, wherein the sentiment analysis module uses an optional large language model (LLM) to enhance detection of emotional stress or cognitive shifts in real-time therapy sessions.
